(19) United States
(12) Patent Application Publication
(10) Pub. No.: US 2002/0192754 A1
(43) Pub. Date: Dec. 19, 2002

(54) Method for producing active serine proteases and inactive variants

(75) Inventors: Dieter Jenne, Neuried (DE); Elke Wilharm, Planegg (DE); Marina A. A. Parry, Arlesheim (CH); Wolfram Bode, Gauting (DE); Robert Huber, Germering (DE)

Correspondence Address: HESLIN ROTHENBERG FARLEY & MESITI PC, 5 COLUMBIA CIRCLE, ALBANY, NY, 12203, US

(73) Assignee: MAX-PLANCK-GESELLSCHAFT E.V., Munchen (DE)

(21) Appl. No.: 10/093,944

(22) Filed: Mar. 8, 2002

Related U.S. Application Data
(63) Continuation of application No. PCT/EP00/08803, filed on Sep. 8, 2000.

(30) Foreign Application Priority Data
Sep. 9, 1999 (DE) DE 199 43 177.9

(51) Int. Cl. C12P 21/02; C12N 1/21; C12N 9/64

(52) U.S. Cl. 435/69.1; 435/226; 435/252.3

(57) ABSTRACT

A method for producing biologically active serine proteases, isolated serine protease domains and their amino acid variants in a prokaryotic host is disclosed. The method comprises an N-terminal addition of a helper sequence consisting of a dipeptide which is suitable for degradation by a dipeptidyl amino peptidase, by the expression of the serine protease(s) and/or its (their) fragments containing the N-terminal dipeptide helper sequence, optionally as inclusion bodies, and by the renaturation of the expressed proteins and the activation of the serine protease(s) and/or serine protease domains by splitting off the helper sequence using an exopeptidase.

### CROSS REFERENCE TO RELATED APPLICATIONS

This application is a continuation of International Patent Application Number PCT/EP00/08803, filed Sep. 8, 2000 and claims priority from German Patent Application Number DE 199 43 177.9, filed Sep. 9, 1999. The entire contents of the earlier applications is incorporated herein by reference.

### FIELD OF THE INVENTION

The invention relates to a method for producing biologically active serine proteases and isolated serine protease domains, as well as enzymatically inactive variants of such serine proteases and serine protease domains in prokaryotic hosts.

### BACKGROUND OF THE INVENTION

Proteases are special proteins having peptidolytic and esterolytic properties and can irreversibly alter and convert other substances and proteins (substrates) catalytically. Corresponding to the functionally relevant molecule groups of the catalytically active center, these proteases can be divided into four major classes: serine dependent proteases, cysteine proteases, aspartases and metalloproteases.

Serine-type proteases are divided into two large families, the family of actual serine proteases and the subtilisine family. The most widely known representatives of the serine proteases are the digestive enzymes of the gastrointestinal tract, trypsin, chymotrypsin and pancreatic elastase, the anti-bacterial and matrix-destroying enzymes of the neutrophilic granulocytes, leukocyte elastase and cathepsin G, the kallikreins of the salivary glands and the serine proteases of the blood clotting and immune system. Serine proteases in secretory granules from mastocytes, lymphocytes, phagocytes or natural killer cells and the serine proteases of the complement system, play a significant role in the immune defense against viruses, parasites, bacteria and tumor cells, and in autoimmune processes.

Serine proteases are specialized for different substrates, and can hydrolyze a peptide bond after aspartic acid groups (granzyme B, induction of DNA fragmentation in lysed target cells), arginine and lysine groups (trypsin, granzyme A and granzyme K), methionine groups (granzyme M, “Met-ase”) or after hydrophobic amino acids (elastase, proteinase 3, pancreatic elastase, chymotrypsin). A series of lymphocyte-specific serine proteases (called granzymes) are secreted in the target cell lysis and directly and indirectly involved in the destruction of target cells by activated killer cells after being absorbed into the cytosol of the target cell. Cathepsin G, proteinase 3 and leukocyte elastase are serine proteases comprising neutrophilic granulocytes, which break down, among other things, elastin and other matrix components, and are considered vital pathogenicity factors in various chronic inflammatory diseases and autoimmune reactions. Proteinase 3 was also identified as the disease-specific autoantigen of Wegener's granulomatosis and could be used in the future in treating patients with this disease.

Although it has long been known that serine proteases are key elements in biological processes such as immune defense, and are therefore of great medical significance, only a few methods for producing these serine proteases on an industrial scale have been described.

For example, Smyth et al., J. Immunol. 154:6299-6305 (1995) and Kummer et al., J. Biol. Chem. 271:9281-9286 (1996), describe the expression of recombinant proteases, but here eukaryotic expression systems are used in which these serine proteases are only produced in small quantities. The efficiency of a biosynthetic production in eukaryotes is unsatisfactory, as are the observed contaminations due to nonfunctional by-products and cell components. The problem is compounded by a complicated purification protocol for separating out homologous cellular proteins, which is usually associated with further losses of the protein to be purified.

Further expression experiments were conducted in a yeast system [Pham et al., J. Biol. Chem., 273:1629-1633 (1998)] and a baculose system [Xia et al., Biochem. Biophys. Res.Com., 243:384-389 (1998)]. Both systems are only suited to a limited extent for representing homogeneously pure serine proteases, since a contamination with undesired eukaryotic proteases also cannot be precluded here. It was also discovered that certain serine proteases can already be activated during biosynthesis in host cells (mammal or insect cell lines) and can thus damage the host cells.

Babé et al., Biotechnol. Appl. Biochem., 27:117-124 (1998), describe an expression method of a serine protease in a prokaryotic system with secretion into the extracellular medium. These authors, however, did not undertake to remold the expressed protein and process it with cathepsin C. Furthermore, only a low yield of 200 μg/l and a short shelf life of the obtained serine protease were observed.

Beresford et al, Proc. Nat'l. Acad. Sci. USA, 94:9285-9290 (1997), in contrast, demonstrated the prokaryotic production of recombinant granzyme A in E. coli, which was obtained in vitro through the cleavage of an enterokinase site introduced in a recombinant process. Here, however, the natural signal peptide sequence must be recombinantly replaced with a bacterial peIB signal sequence so that an inactive proenzyme can be exported into the bacterial periplasma. However, as mentioned in Xia et al., this method could not be used to produce a similar protease, granzyme B.

Höpfner et al., EMBO J.,16:6626-6635 (1997), present the expression of an active serine protease in E. coli, but in this method the activation must be effected with Russel's Viper Venom. The very sequence-specific enzyme (an endoproteolytically active serine protease) contained in this poison, however, is not readily available. Moreover, the signal sequence recognized by the protease is considerably longer than and different from the naturally occurring propeptides, which could reduce the effectiveness of the renaturation.

U.S. Pat. No. 5,679,552 describes the generation of biologically active proteins whose correct N-terminus is attained through limited proteolysis of an N-terminal helper sequence using cathepsin C. The exact processing of the even numbered amino acid helper sequence with the aid of cathepsin C is achieved in that so-called cathepsin C stop sequences are artificially inserted into the amino terminus of the protein to be produced. The method is limited, however, to proteins possessing certain sequence properties at the mature N-terminus, i.e., proteins with lysine or arginine in the first position or proteins having proline in the second or third position inside the sequence of the protein to be produced. Serine proteases having the N-terminal consensus sequence Ile-(Ile/Val)-Gly-Gly cannot be produced with the method described in U.S. Pat. No. 5,679,552. The highly conservative N-terminal sequence of functionally active serine proteases does not correspond to any of the known cathepsin C stop sequences.

EP 0 397 420 discloses the enzymatic conversion of recombinant proteins with helper sequences using cathepsin C and a novel N-terminal stop sequence (Met-Tyr and Met-AEG). The N-terminal end (Met-Tyr or Met-AEG) of the recombinant protein to be produced, which is described for the first time in this patent document, cannot be cleaved by exopeptidases such as cathepsin C. The stop sequences presented in EP 0 397 420 are likewise unsuitable for processing and representing a functional N-terminus in catalytically active serine proteases.

U.S. Pat. No. 4,861,868 describes the generation of proteins with alanine at the N-terminus after cleaving one or more methionines with a monoamino exopeptidase from E. coli, the methionine amino peptidase. An N-terminal alanine is, however, unsuitable for representing an active serine protease, because the highly conservative N-terminal Ile-(Ile/Val)-Gly-Gly is essential for attaining the active conformation.

U.S. Pat. No. 5,013,662 describes the production of N-terminal methionine-free proteins in E. coli. The N-terminal methionine required by the initiation codon is cleaved by methionine amino peptidase in vitro or in vivo. The N-termini of serine proteases (Ile-(Ile/Val)-Gly-Gly) also cannot be produced with this method, because the methionine amino peptidase methionine can only be cleaved before small amino acids (Gly, Val, Ser, Ala), but not large, aliphatic amino acids, such as the absolutely necessary isoleucine.

E. Wilharm and D. Jenne reported that a tripeptide Met-Gly-Glu could be used to produce catalytically active serine proteases. N-terminal methionine is quantitatively removed before glycine in endogenetically-expressed E. coli proteins. The remaining dipeptide, the component of the natural granzyme B sequence, should be cleaved by cathepsin C.

This method has not been proven effective, however. In overexpression in E. coli, even under the best cultural conditions, only about 60 to 70% of all methionine is removed. The conversion of the methionine-free proform by cathepsin C is also incomplete. The obtained products are analyzed through sequencing and identified as, among other things, Met-Gly-Glu-GzmK, Gly-Glu-GzmK and (completely processed mature) GzmK molecules. The result was an intolerable product heterogeneity that is unacceptable in numerous applications.

It is therefore desirable to provide a method that can be used for producing biologically active serine proteases or serine protease domains and catalytically inactive, but correctly folded, variants in prokaryotic hosts. The present invention provides such a method.

The method was not developed for a direct synthesis of natural serine proteases with a complex domain composition (blood clotting factors, complement proteases), but especially for simple serine proteases that comprise a single domain, the catalytic domain, and, precisely for this reason, perform a specific peptidolytic or esterolytic function with a natural or artificial specificity (designer activities). These simple serine proteases are present in diverse forms in nature and perform a broad range of tasks in the area of cellular and humoral immune defense (mastocyte, granulocyte and lymphocyte proteases, complement factor D), gastroenteric digestion (trypsins, chymotrypsin and elastases), exocrine and endocrine organs (kallikreins), and for the normal physiology of the skin and nervous system (neuropsin, kallikrein homologues).

The method is suitable for mass-producing these serine proteases and serine proteases derived from them with an artificial substrate specificity. The products can therefore be produced economically in unlimited quantities as research reagents, therapeutic substances and for the development and testing of inhibitors in vivo.

### SUMMARY OF THE INVENTION

The principals of the present invention, in contrast to the state of the art, provide a method that, for terminating exopeptidase reactions, requires no specific, linear stop sequences at the N-terminus of the proteins to be produced, and an N-terminal helper sequence is cleaved selectively and completely, despite errors in a known stop sequence for exopeptidases.

Experiments regarding the function and specificity of cathepsin C revealed that the naturally present, strongly conservative N-terminus of serine proteases and serine protease domains is resistant to an exopeptidolytic degradation, e.g., due to cathepsin C in vitro and thus can be used as a new stop sequence for processing zymogens in active serine proteases with the aid of cathepsin C or similar exopeptidases if a homogeneous initial product is present and this initial product supports a dipeptide helper sequence before the beginning of the mature enzyme.

Hence, the present invention relates to a method for producing biologically active serine proteases, isolated serine protease domains and their amino acid variants in a prokaryotic host, the method comprising the steps of:

(a) the N-terminal addition of a helper sequence comprising a dipeptide that is suitable for degradation by a dipeptidylamino peptidase;

(b) the expression of an artificial nucleotide sequence having the characteristics of folding similar to the corresponding, natural serine proteases and forming a stable three-dimensional structure;

(c) the expression of the serine protease(s) and/or its (their) fragments with an N-terminal dipeptide helper sequence, possibly as inclusion bodies;

(f) the renaturation of the expressed proteins; and

(g) the activation of the serine protease(s) and/or a serine protease domain through the splitting of the helper sequence by an exopeptidase.

In connection with the present invention, serine protease refers to all proteins that are structurally similar to trypsin and chymotrypsin. These proteins include, for example, the serine proteases of the blood clotting and complement system, immune defense cells, the gastrointestinal tract and the exocrine glands. The term serine protease domain refers to independently folding parts of complexly structured proteins demonstrating a structural, three-dimensional similarity to serine proteases. These serine protease domains predominantly exhibit peptidolytic and esterolytic properties, but occasionally perform other functions as well. The serine proteases of the blood clotting and complement system comprise different covalently bonded protein domains and a carboxy-terminal-localized serine protease domain having catalytic properties. A critical factor for the activation and thermodynamically stable folding of serine proteases is a correctly processed N-terminus, which generally begins with an isoleucine or valine.

Prokaryotic hosts that can be used within the scope of the invention are known to specialists and include organisms such as Escherichia, bacillus, Erwinia and Serratia species, particularly E. coli, bacillus subtilis, Erwinia chrysanthemi, Erwinia carotovora or Serratia marcescens. E. coli and bacillus subtilis are preferably used here.

Conventional molecular biological techniques can be used to execute the biotechnological method of the invention for obtaining and activating correctly folded, functionally active serine proteases [see, for example, Sambrook et al., Molecular Cloning, a laboratory manual, 2^{nd}. Ed., Cold Spring Harbor, N.Y. (1998) or (1998) Ausubel et al., Current protocols in molecular biology, Current Protocols, Vols. 1 and 2 (1994)].

The inventive addition of a helper sequence at the amino terminus of serine proteases or serine protease domains is effected with the aid of cloning techniques and gene manipulations in prokaryotic cells; here, DNA molecules or parts of these molecules are introduced into plasmids and possibly adapted to the necessary sequence through targeted mutagenesis and the recombination of DNA segments. Thus, standard methods such as the polymerase chain reaction (PCR) and ligation reactions can be employed to create vector constructs that lead to the expression of serine proteases with N-terminal helper sequences. These standard methods, described, for example, in Sambrook et al. and Mullis et al., The Polymerase Chain Reaction, Birkhäuser, Boston (1994), not only permit the cloning and expression of heterological proteins at the cDNA level, but also the purposeful exchange of bases and the addition of natural or synthetic sequences. In this way, known molecular biological methods can be implemented in the use of the described method for producing sequence-modified serine proteases having novel properties in prokaryotic hosts.

For linking DNA fragments, adaptors or linkers can be joined to the fragments to be cloned. Moreover, appropriate restriction interfaces can be joined or excess non-coding DNA or undesired restriction interfaces can be removed. If insertions, deletions or substitutions are desired, the techniques of in vitro mutagenesis, repair with the aid of modified primers, PCR, restriction digestion and ligation are used. The degeneration of the genetic code offers specialists the option of adapting the nucleotide sequence of the DNA sequence to the codon preference of the respective prokaryotic host. Restriction digestion, sequencing and further biochemical-molecular biological tests are required as analytic methods for assessing the work results.

The desired sequence to be expressed can be produced synthetically, or obtained naturally or contain a combination of synthetic and natural DNA components. Generally, synthetic DNA sequences are generated with codons that are preferred by prokaryotes. These prokaryote-preferred codons can be taken from published tables (e.g., http://pegasus.uthct.edu), and are found most commonly in strongly expressed endogenetic proteins. In the preparation of the expression cassette, various DNA sub-fragments can be individually manipulated and combined to obtain a DNA sequence that is equipped with a correct reading raster and is translated in the correct direction. In this case as well, adaptors or linkers can be used to simplify the process of linking DNA fragments.

To perform an N-terminal addition of helper sequences for serine proteases, as mentioned above, molecular biological DNA vectors with special control ranges are used; these control the transcription of the expression cassette in prokaryotic cells. These control ranges generally include the promoter and special regulatory elements. The regulatory elements, such as the tac-lac, T7 or trp promoter, are well known to specialists. Corresponding prokaryotic expression vectors can be obtained from numerous companies: pET24c and other pET vectors from Novagen; lambda gt11 and pGBT9 from Clontech; and pGX from Qiagen.

A specialist understands that, within the scope of the invention, “expression of the serine protease(s)” means the expression of a heterologous fusion protein in the prokaryotic host. The method for producing serine proteases and/or their fragments includes the expression of a proform in the cytosol of the prokaryotic host, possibly and preferably as inclusion bodies (“IB”). The inclusion body formation depends primarily on the expression rate; there is no definitive correlation between size, hydrophobicity and other characteristics of the protein to be expressed [Lilie, H. et al., Current Opinion in Biotechnology, 9:497-501 (1998)].

At low expression temperatures and with small, hydrophilic proteins, however, the solubility of the recombinant protein is an issue, e.g., Alkalische Phosphatase [Alkaline Phosphatase] [Derman et al., Science 262:1744-1747 (1993); Proba et al., “Gene,” Genes, 159:203-207 (1995)]. A natural conformation can also be attained for recombinant proteins whose three-dimensional folding is not decisively stabilized by the formation of disulfide bridges (often found in zytoplasmatic proteins). In E. coli with a thioredoxin reductase deficiency (e.g. Novagen AD494 DE 3), the cytoplasm is less reductive and in a few cases even permits the formation of disulfide bridges (Derman et al.).

In addition to the expression of the heterologous fusion proteins of the serine proteases and/or their fragments as inclusion bodies, the invention also encompasses a method in which the expression is performed as soluble recombinant protein/peptides. The term “renaturation of the expressed fusion proteins” within the scope of the invention refers to the solubilization of protein aggregates and folding in three-dimensional structures that mimic natural ones and are stable in physiological buffer solutions.

The method according to the invention also includes the activation of the serine proteases to be produced, or their serine protease domains, through the cleavage of a suitable helper sequence by an exopeptidase. Exopeptidases are subdivided into diamino peptidases and monoamino peptidases, which cleave two or one amino acid(s) in every processing step. Diamino (exo)peptidases are particularly preferred, as will be described in detail below.

Examples include: cathepsin C; cathepsin W; cathepsin B or diamino peptidase IV; cathepsin C-like functional homologues in other species such as in dictyolstelium discoideum and C. elegans. A characteristic feature of the exopeptidase substrates to be processed is that they do not support the known exopeptidase stop sequences in the region of the helper sequence or in the amino terminal region of the desired product. A sequential conversion could be executed with an advantageous combination of different exopeptidases, with methionine being cleaved in a first step with a specific methionine amino peptidase and dipeptide(s) being removed in subsequent steps.

In a preferred embodiment of the method according to the invention, dipeptides or a combination of several different dipeptides from a pool of suitable dipeptides without natural stop sequences are additionally positioned before the N-terminus of the serine protease to be expressed. These peptide helper sequences include any amino acid combination, but proline, lysine or arginine cannot occupy the first position of this peptide, and proline cannot occupy the second position.

In an especially preferred embodiment of the method according to the invention, in step (a) a dipeptide of the form Met-X, for example Met-Glu, is added before the sequence of the desired serine protease product; in step (g), the dipeptide is cleaved in a self-limiting conversion with the use of cathepsin C. In this especially preferred embodiment of the method according to the invention, the dipeptide joined to the N-terminus begins with a methionine. Because translation begins with a formyl-methionine group in prokaryotes, which is removed in some cases by the endogenetic E. coli formylase in connection with the E. coli methionine amino peptidase in the cytosol of the bacteria, it was necessary to identify a suitable, universally applicable propeptide that cannot be changed into E. coli during biosynthesis, on the one hand, but can be selectively enzymatically cleaved in a simple, efficient manner, without attacking the recombinant protein, after the precursor product has been refolded. Thus, the methionine-containing dipeptides (Met-Y) that are added in this preferred embodiment are resistant to post-translational processing into E. coli, yet are a good substrate for dipeptidyl amino peptidases that have only been verified in eukaryotes to this point.

As described above, a sequence comprising a plurality of Met groups can also be joined before the helper dipeptide to be cleaved. In other words, sequences of the form (Met)n-Glu, such as Met-Met-Gly (where n can represent a natural number up to 40) can be used. These methionines can be cleaved during or following the expression, that is, in vitro, by methionine amino peptidase(s).

According to the invention, however, it must be noted that the cleavage of the methionine(s) leaves a helper sequence with an even number of amino acids without stop amino acids. If an odd number of amino acids were obtained after the cleavage of the initial methionine(s), the use of dipeptidyl amino peptidases could no longer assure the generation of an exact N-terminus of the serine protease(s) to be produced and/or its (their) fragments.

Therefore, in accordance with the invention, it is practical to combine a methionine aminopeptidase with a diamino exopeptidase if additional methionines joined before the dipeptide helper sequence must be removed.

The dipeptide helper sequence Met-Glu is particularly well suited for the embodiment proposed above; individual methionines or groups of methionines can be positioned before this dipeptide helper sequence. Proline at the second position in the dipeptide should be avoided, because this prevents the cleavage of the dipeptide by individual exopeptidases such as cathepsin C. It should be pointed out here that tyrosine or arginine at the second position following a methionine should be avoided if cathepsin C is to be used as an exopeptidase in the method of the invention.

In a particularly preferred embodiment, the dipeptide Met-Glu is used as the helper sequence and cathepsin C is used as the conversion enzyme.

The present invention thus also relates to a method in which the exopeptidase is a monoamino peptidase and/or a diamino peptidase in the preferred embodiment. Monoamino peptidase refers to, among others, the aforementioned methionine amino peptidase from E. coli [(Ben-Bassat et al., J. Bacteriol., 169:751-757 (1987)]. In an especially preferred embodiment, the diamino exopeptidase is cathepsin C or a cathepsin C homologue (e.g., RCP, described in U.S. Pat. No. 5,637,462, the entire contents of which is incorporated herein by reference).

The invention further relates to a method as described above, in which the biologically active serine proteases and serine protease domains to be produced do not inactivate the exopeptidase to be used and, in a further embodiment, the exopeptidase does not irreversibly alter the protein to be produced. The inactivation of the exopeptidase(s) by active serine proteases (e.g., through proteolytic cleavage) should be avoided. Within the scope of the invention, an irreversible change in the serine protease(s) to be produced and/or its (their) fragments means cleavage, conformation changes and/or inhibitions. Also within the scope of the invention, aggregate formation between the proteins/fragments to be produced and the used exopeptidase should be avoided.

In a further embodiment, the invention encompasses a method in which the serine protease fragment to be produced is the catalytic domain of a serine protease.

The invention further encompasses a method in which the biologically active serine protease to be produced comprises one or more non-covalently-bonded catalytic domains.

The invention also encompasses those serine proteases and their fragments that appear naturally as catalytically inactive serine protease variants due to amino acid substitutions and/or amino acid insertions and perform other noncatalytic tasks.

The invention also encompasses serine proteases formed through mutagenesis without catalytic activity.

In a preferred embodiment, the invention encompasses a method in which the serine protease is leukocyte-elastase, proteinase 3, complement factor D, azurocidine, mastocyte chymase, pancreatic trypsin, pancreatic chymotrypsin, mastocyte tryptase, glandular kallikrein, cathepsin G, a granzyme, or a catalytic domain of complement proteases or blood clotting proteases.

In a particularly preferred embodiment, the invention encompasses a method for producing a granzyme, the granzyme being granzyme A, B, K, H, M or L. Granzyme L includes the nucleotide sequence presented in SEQ ID No. 1.

These and other embodiments are known to specialists by the description and the examples of the present invention. More detailed literature relating to one of the methods described above, products and therapeutic applications of these products which can be produced with the implementation of the present invention, can be found in public libraries with the aid of electronic search devices. To this end, Internet-accessible public databases such as “Medline” (http://www.ncbi.nlm.nih.gov/PubMed/medline.html) are helpful. Further databases and addresses are known to those of skill in the art and can be accessed via the Internet, for example, via http://www.lycos.com. An overview of sources and information on patents and patent applications in the field of biotechnology is given in Berks, TIBTECH, 12:352-364 (1994).

The entire contents of each state of the art references cited herein is incorporated fully into this disclosure by reference.

### BRIEF DESCRIPTION OF THE DRAWINGS

The invention will now be described in detail by way of the following examples, experimental details and drawings, wherein:

FIG. 1 is a description of the Primers used

FIG. 2a is an analysis of the Expression, Removal and Renaturation of Human Granzyme K. Coomassie-colored 12% SDS gel with lysate of a noninduced culture (trace 1), lysate of an IPTG-induced culture (trace 2), preparation of the inclusion bodies from the induced lysate (trace 3), renatured zymogen after ion exchange chromatography (trace 4) and native human granzyme K after activation with cathepsin C and ion-exchange chromatography (trace 5).

FIG. 2b is an analysis of the expression of Human Granzyme M (hGzmM) with the use of different expression constructs. 12% SDS-PAGE with subsequent Coomassie coloring of hGzmM with a natural C-terminus (referred to as hGzmM/WT) and (His)₈-Strep-tag-C-terminus (referred to as hGzmM/IIST) and of hGzmK as the expression control. The digestion procedures are: 1) noninduced culture, digestion in 2.5% SDS, 5% b-thiolethanol; 2) induced culture, digestion as in 1; 3) induced culture, digestion in 5% SDS, 200 mM DTT, 5% b-thiolethanol; and 4) induced culture with idealized codons, digestion as in 1.

FIG. 3 shows the N-Terminal sequence comparison between Human Granzyme K (hGzmK) and Human Granzyme M (hGzmM) at the amino acid level (A) and nucleotide level (B). The codon frequencies in E. coli (Ausubel et al.) re listed in percentages; rare codons are underlined. The oligo proposed for the N-terminal optimization of the expression constructs is shown in (C); the altered positions are underlined.

FIG. 4a shows the substrate specificity of Human Granzyme K (hGzmK). Respectively 0.1 mM of the thiobenzylester substrates and respectively 3 nM of the proform (black), the converted form (hatched) and the converted S195A mutants (gray) were used. The substrate conversion was measured over 5 minutes at 405 nm and room temperature.

FIG. 4b shows the substrate specificity of Mouse Granzyme K (mgzmK). Respectively 0.1 mM of the thiobenzylester substrates and respectively 5 nM of the proform (black) and the converted form (hatched) were used. The substrate conversion was measured over 5 minutes at 405 nm and room temperature.

FIG. 4c shows the substrate specificity of Human Granzyme M (hGzmM). Respectively 0.1 mM of the thiobenzylester substrates and respectively 15 nM of the proform (black), the converted form (hatched) and the converted S195A mutants (gray) were used. The substrate conversion was measured over 5 minutes at 405 nm and room temperature.

FIG. 5 provides the cDNA Sequence of Granzyme L

FIG. 6 provides the Amino acid Sequence of Granzyme L

FIG. 7 illustrates the construction of Human GzmK Precursors in the Plasmid Vector pET24c. The expression cassettes for human GzmK precursors were cloned into the Nde I and Eco RI interfaces of pET24c(+) (Invitrogen). Transcription from the T7 promoter (black arrow) is driven by chromosomally coded T7-RNA polymerase, which can be induced by isopropyl-I-thio-Øb-D-galactopyranoside. Three constructs having amino terminal sequence extensions (open bar) at the amino terminus of mature GzmK (gray bar) were produced. Removal of the amino terminal pre-sequences was achieved with an endogenetic bacterial MAP (construct A), cathepsin C after renaturation (construct C), or a combination of MAP and cathepsin C after the refolding of GzmK precursors (construct B).

FIG. 8 illustrates the preparation of catalytically active Human GzmK from E. coli Inclusion Bodies. Noninduced and induced bacterial cell lysate (traces 1 and 2), purified inclusion bodies (trace 3) and refolded GzmK precursors before and after Met-Glu removal with the use of cathepsin C (traces 4 and 5) were made visible with Coomassie brilliant blue coloring in accordance with SDS-PAGE.

FIG. 9 shows the substrate specificity of Recombinant Human GzmK. Enzymatic activity of unprocessed zymogen (M-E pre-sequence, black bars) and activated GzmK (hatched bars) was measured with the aid of the given thiobenzylester substrates with an end concentration of 0.1 nM. The enzyme concentration was 3 nM for both the unprocessed and the activated forms of GzmK.

FIG. 10 shows the inhibitory effect of human blood plasma on GzmK Activity in the absence (upper figure segment) or presence (lower figure segment) of 0.5 Units of Heparin/ml. The Z-Lys-Sbzl activity of 3 nM GzmK was measured after 15-minute incubation with increasing quantities of human EDTA plasma, as shown on the x-axis. The net activity (black bars) of recombinant GzmK and the background activity of plasma dilutions (hatched bars) with standard errors of triple measurements are indicated by respective columns.

FIG. 11 shows the inhibition of GzmK through purified IaI, Bicunin D2 and ATIII in comparison to 2.5% human plasma. GzmK activity (3 nM, first to seventh columns) was measured after incubation with 2.5% (V/V) human blood plasma (second column), 67.5 nM ATIII (third column), 26 nM bicunin D2 (fourth column), a mixture of 2.5 nM bicunin D2 and 23.2 nM IaI (fifth column), 26 nM bicunin D2 and 67.5 nM IaI (seventh column). Inhibitor concentrations were selected such that physiological ATIII, total bicunin and total IaI concentrations of human plasma diluted 40 times were simulated. The molar ratios (I:E) between ATIII (third, sixth and seventh columns), bicunin D2 (fourth and sixth columns), mixtures of IaI and bicunin D2 (fifth and seventh columns) and GzmK were 22.5, 8.5 and 8.5 (7.7+0.8), respectively. The average percentage of remaining Z-Lys-SBzl activity with its standard errors from three experiments is shown as a black column with error bars.

FIG. 12 shows the effect of polyclonal IgG antibodies against IaI, Bicunin and ATIII on the inhibition of GzmK. In the upper figure section, inhibition of GzmK in the presence of monospecific antibodies against purified inhibitors (black bars) was compared to those without neutralizing antibodies (hatched bars). Bicunin D2 (30 nM), IaI (76 nM), and ATIII (125 nM) were preincubated for 15 minutes prior to the addition of 3 nM GzmK with monospecific antibodies. Inhibitor-enzyme relations (I:E) are shown beneath the upper figure segment. The lower figure segment shows that the same quantity of polyclonal antibodies having a specificity for bicunin (second column), IaI (third column), H chains of IaI (fourth column) or ATIII (fifth column) was mixed with a 2.5% plasma dilution and pre-incubated for 15 minutes prior to the addition of 3 nM GzmK. The remaining GzmK activity was ascertained 15 minutes later. Percentage values for Z-Lys-SBzl activity are given with standard errors. Each antibody was affinity-purified via protein G sepharose and used in an end concentration of 120 μg/ml. Only bicunin and IaI antibodies neutralize the inhibitory effect of human whole plasma on GzmK.

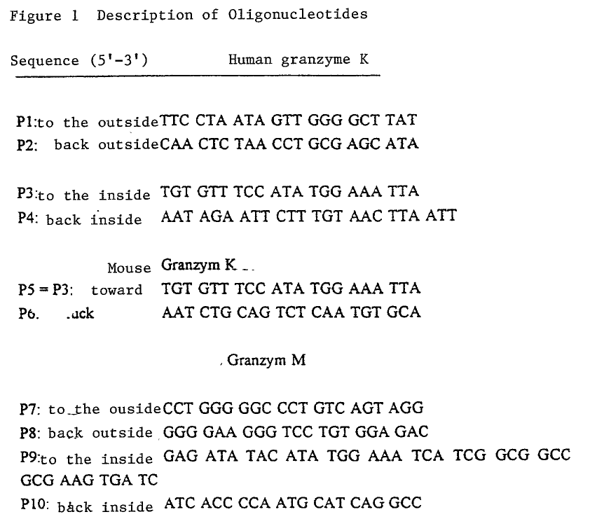

### DETAILED DESCRIPTION OF THE INVENTION

### EXAMPLE 1

Construction of the Expression Cassette

1. In this embodiment, the expression cassette was constructed with the addition of the N-terminal helper sequence Met-Glu (using the example of human granzyme K and mouse granzyme K).

For cloning human granzyme K, the vector pET24c (Novagen) was spliced with the the restriction endonucleases NdeI and EcoRI. The sequence that codes for human granzyme K was amplified by means of a two-stage PCR of human bone marrow cDNA.

In the first PCR round, the human granzyme K-cDNA was amplified between the center of the first and the end of the fifth exons of human bone marrow cDNA with a pair of correctly hybridizing oligonucleotides (so-called outer oligos—see P1 and P2 in the sequence protocol). PCR conditions: matrix DNA: 1 μl of an mRNA (2 μg) rewritten with reverse transcriptase into cDNA; nucleotide: respectively 0.2 mM; oligos; respectively 1 μM; enzyme 0.5 μl/50 μl batch [2.5 units/μl] of native Pfu polymerase (Stratagene); buffer: Stratagene; program: non-cyclical denaturing: 5 minutes at 95° C., cyclical steps: 1 minute at 95° C., 1 minute at 56° C., 1 minute at 72° C., 35 cycles, nonecyclical elongation: 5 minutes at 72° C.

The obtained PCR product served as the matrix sequence in the second PCR round, in which the insert for the cloning was amplified in the pET24c vector by means of a second oligonucleotide pair (inner oligos—see P3 and P4 in the sequence protocol). PCR conditions: matrix DNA: 5 ng; oligos: respectively 1 μM; nucleotide: respectively 0.2 mM; enzyme: 0.5 μl/50 μl batch [2.5 units/μl] of native Pfu polymerase (Stratagene); buffer: Stratagene; program: non-cyclical denaturing: 5 minutes at 95° C., cyclical steps: 1 minute at 95° C., 1 minute at 58° C., 1 minute at 72° C., 24 cycles, noncyclical elongation: 5 minutes at 72° C.

The NdeI and EcoRI interfaces were inserted into the oligos. The amplified substance obtained in this manner was removed from the gel (Qiaquick protocol from Qiagen), spliced with the restriction endonucleases NdeI and EcoRI and ligated into the vector so the translation of the transcript begins with the methionine of the NdeI palindrome 8 bases in 3′ direction from the ribosomal binding site. Ligation conditions: enzyme: 1.5 μl/20 μl batch [1 unit/μl] T4 ligase (Boehringer Mannheim); vector: 50 ng/20 μl batch; insert: 50 ng/20 μl batch; buffer: Boehringer Mannheim; incubation: 16 hours at 15° C. The N-terminal sequence for both granzymes comprises the pro(di)peptide Met-Glu and the adjoining conserved sequence of the mature granzyme Ile-Ile-Gly-Gly. At the 3′ end, the translation halts with the natural stop codon.

The mouse granzyme K was amplified with the use of the identical oligonucleotide in the N-terminal region (P5 or P3) and P6 as the backward primer. Starting with mouse splenic cDNA, the granzyme K fragment of the mouse was amplified for the expression cassette in 35 cycles with the use of 5 ng DNA, removed from the gel (Qiaquick protocol by Qiagen) and spliced with the restriction endonuclease NdeI, and the 3′ end was kinased. For this PCR, 0.2 mM of the four nucleotides, 1 μM of each oligo and 0.5 μl/50 μl batch [2.5 units/μl] of native Pfu polymerase (stratagene) were used in the buffer system of stratagene, and the following thermocycler program was employed: noncyclical denaturing: 5 minutes at 95° C., cyclical work steps: 1 minute at 95° C., 1 minute at 51° C., 1 minute at 72° C., 35 cycles, non-cyclical elongation: 5 minutes at 72° C. For cloning in pET24c, the vector was opened through digestion with HindIII and the overhang was filled, resulting in a smooth end and the linearized vector was subsequently spliced with the restriction enzyme NdeI. The insert was ligated into the prepared vector as described above.

The resulting clones were selectioned on canamycin [30 μg/ml] and verified through restriction analysis (double digestion) with the aid of NdeI and XhoI (New England Biolabs) and sequencing.

2. In this embodiment, the expression cassette was constructed with sequence-neutral codon optimization (using the example of human granzyme M).

The vector pET24c-His-Strep-tag (modified Novagen vector) was spliced with the restriction endonucleases NdeI and PstI for cloning human granzyme M. The sequence that codes for human granzyme M was amplified by means of a two-stage PCR of human bone marrow cDNA. In the first PCR round, the human granzyme M-cDNA was amplified between the center of the first and the end of the fifth exons of human bone marrow cDNA with a pair of correctly-hybridizing oligonucleotides (so-called outer oligos—see P7 and P8 in the sequence protocol). PCR conditions: matrix DNA: 1 μl of an mRNA (2 μg) rewritten with reverse transcriptase into cDNA; nucleotide: respectively 0.2 mM; oligos; respectively 1 μM; enzyme 0.5 μl/50 μl batch [2.5 units/μl] of native Pfu polymerase (Stratagene); buffer: Stratagene; program: non-cyclical denaturing: 5 minutes at 95° C., cyclical: 1 minute at 95° C., 1 minute at 56° C., 1 minute at 72° C., 24 cycles, noncyclical elongation: 5 minutes at 72° C.

The obtained PCR product served as the matrix DNA in the second PCR round, in which the insert for cloning was amplified into the pET24c-His-Strep-tag vector by means of a second oligonucleotide pair (inner oligos—see P9 and P10 in the sequence protocol). PCR conditions: matrix DNA: 5 ng; oligos: respectively 1 μM; nucleotide: respectively 0.2 MM; enzyme: 0.5 μl/50 μl batch [2.5 units/μl] of native Pfu polymerase (Stratagene); buffer: Stratagene; program: non-cyclical denaturing: 5 minutes at 95° C., cyclical: 1 minute at 95° C., 1 minute at 58° C., 1 minute at 72° C., 24 cycles, non-cyclical elongation: 5 minutes at 72° C.

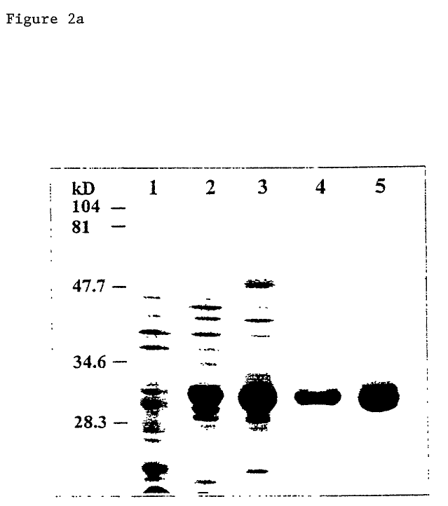

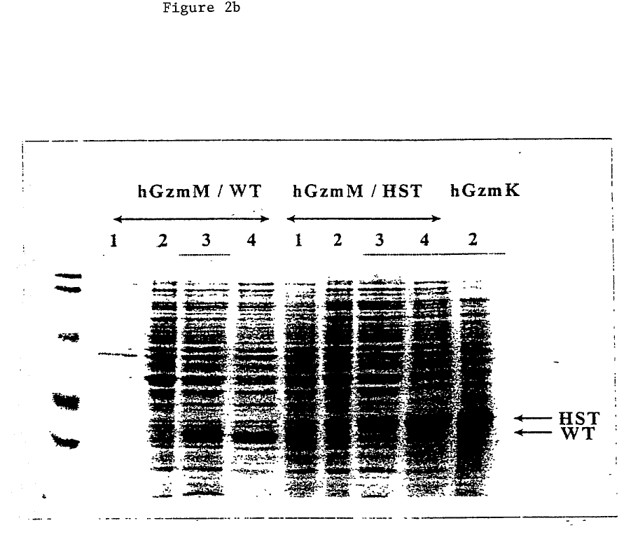

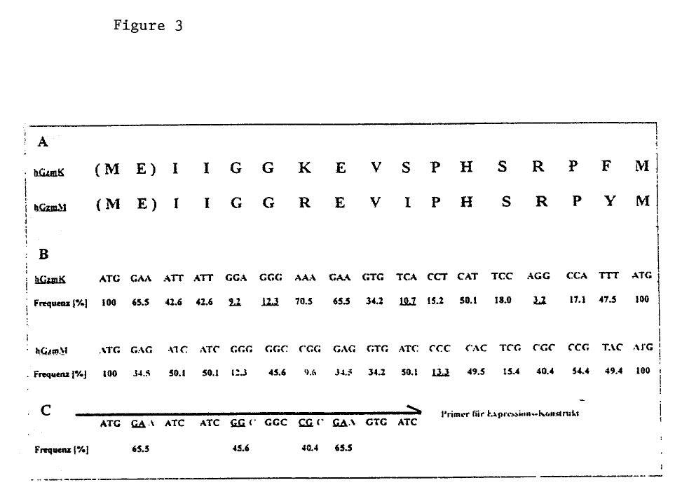

The NdeI and NsiI interfaces were inserted into the oligos. In the oligo P9, the sequence that codes for the first ten amino acids was additionally optimized with respect to the codon frequency in E. coli (FIG. 3). FIG. 2b illustrates the influence of this codon optimization on the expression intensity. The amplified product obtained in this manner was removed from the gel (Qiaquick protocol by Qiagen) and ligated into the vector (ligation conditions: see Example 1a). The pro(di)peptide Met-Glu was also used as the N-terminal helper sequence for mouse granzyme K, and the cDNA reading frame of the mature murine granzyme K was attached thereto. The translation was halted with the natural stop codon at the 3′ end.

The resulting clones were selectioned on canamycin [30 μg/ml] and confirmed through restriction analysis (double digestion) with NdeI and EcoRI (New England Biolabs) and DNA sequencing.

### EXAMPLE 2

### Fermentation, Preparation of “Inclusion Bodies” and Their Solubilization

(Expression of a Serine Protease as Inclusion Bodies)

The plasmids constructed in accordance with Examples 1a and 1b were transformed into the expression stem E. coli B834 DE3 (Novagen), and the expression was first tested on a small scale. The 10 ml cultures were drawn in with LB canamycin (for concentration, see Example 1) to an OD₆₀₀ of 0.5; the expression was induced with 1 mM IPTG and incubated for 3 hours at 37° C. up to an OD₆₀₀ of 1.5. IPTG activates the lacUV promoter, which controls the chromosomally coded T7 polymerase in the B834 DE3 stem, which in turn takes control of the transcription of the cloned granzyme gene under the T7lac promoter.

A total cell lysate was produced from 50 μl (=0.075 OD) of the induced culture and a non-induced control, and was analyzed by means of SDS-PAGE.

From highly-expressive clones (clear bands at 25 kD in the Coomassie-colored SDS gel—FIGS. 2a and 2b), the culture was repeated on a large scale and an IB preparation was obtained from the total cell lysate.

The following buffers were used to prepare inclusion bodies (“inclusion bodies”):

| | | |
|---|---|---|
| (a) Lysis buffer: | 50 | mM Tris |
| | 10 | μg/ml Dnase |
| | 2 | mM MgCl₂ |
| | 0.25 | mg/ml lysozyme |
| | pH 7.2 | |
| (b) Washing buffer I: | 50 | mM Tris |
| | 60 | mM EDTA |
| | 1.5 | M NaCl |
| | 6% | Triton-X-100 |
| | pH 7.2 | |
| (c) Washing buffer II: | 50 | mM Tris |
| | 60 | mM EDTA |
| | pH 7.2 | |

The bacteria were harvested through centrifuging, and the pellet was washed once with PBS pH 7.4 before being digested in the lysis buffer at room temperature. The bacterial membranes were either broken by two French press cycles (1000-1200 psi) or three sonification cycles (15 minutes each at 320 W); the lysate was mixed with one-third of the volume of the washing buffer I and incubated at room temperature in the overhead shaking machine for one hour. The suspension was centrifuged at 17,2000 g at 4° C. for 20 minutes; the pellet was re suspended in the washing buffer I, incubated for 1 hour at room temperature in the shaking machine and centrifuged again. This procedure was repeated twice with the washing buffer I and three times with the washing buffer II. Following the last centrifuge process, a small aliquot of the IB preparation was analyzed for purity through SDS-PAGE; the remainder was solubilized.

The following buffers were used:

| | | |
|---|---|---|
| (d) Solubilization buffer: | 6 | M guanidinium chloride |
| | 100 | mM Tris |
| | 20 | mM EDTA |
| | 15 | mM GSH |
| | 150 | mM GSSG |
| | pH 8.0 | |
| (e) Dialysis buffer: | 6 | M guanidinium chloride |
| | pH 5.0 | |

About 2 g IB (volume weight with humidity) were re-suspended in the buffer and incubated overnight at room temperature in the shaking machine. Insoluble components were centrifuged off, and this protein solution was subsequently dialyzed against 20 volumes of dialysis buffer for 24 hours at 4° C., with three buffer changes one every 8 hours.

### EXAMPLE 3

Renaturation of the Expressed Proteins

The following buffers were used for the renaturation:

| | | |
|---|---|---|
| (f) Renaturation buffer: | 50 | mM Tris |
| | 0.5 | M arginine |
| | 20 | mM CaCl₂ |
| | 1 | mM EDTA |
| | 0.1 | MNaCl |
| | 0.5 | mM cysteine |
| | pH 8.5 | |
| (g) Dialysis buffer: | PBS | |
| | pH 7.0 | |

The renaturation was effected in three pulses with time intervals of 8 hours each. The renaturation batches of human granzyme K were incubated at room temperature; the mouse granzyme K and human granzyme M were incubated at 4° C. The protein solution (˜10 mg/ml) from Example 2 was diluted 1:100 (Vol/Vol) in the renaturation buffer while being stirred, and incubated without agitation until the next pulse. After the third addition of the protein solution, the re-folding batch was incubated for two more days without agitation at room temperature or 4° C. After the renaturation, the reaction volume was filtered (over cellulose acetate) to a concentration of approximately 50 ml, and dialyzed at 4° C. until the arginine was removed (control via conductivity).

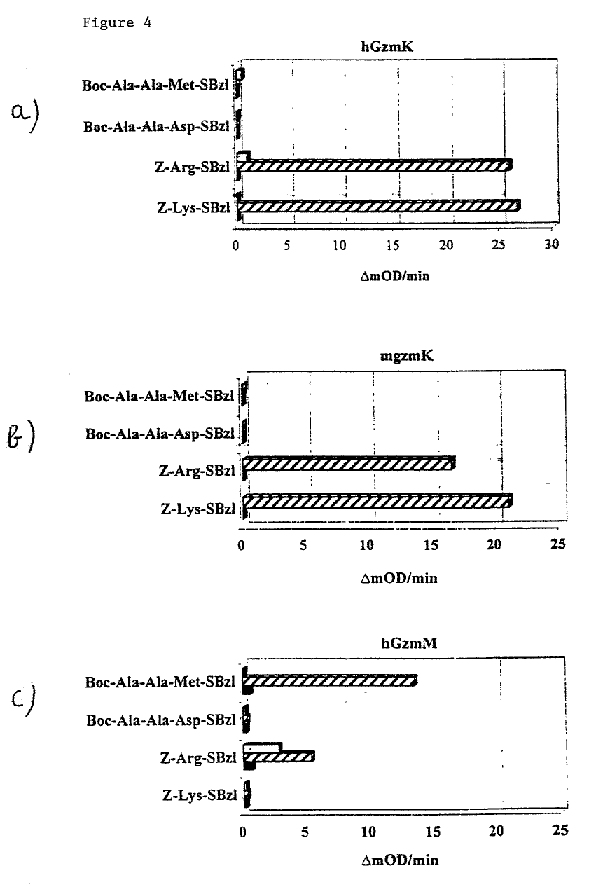

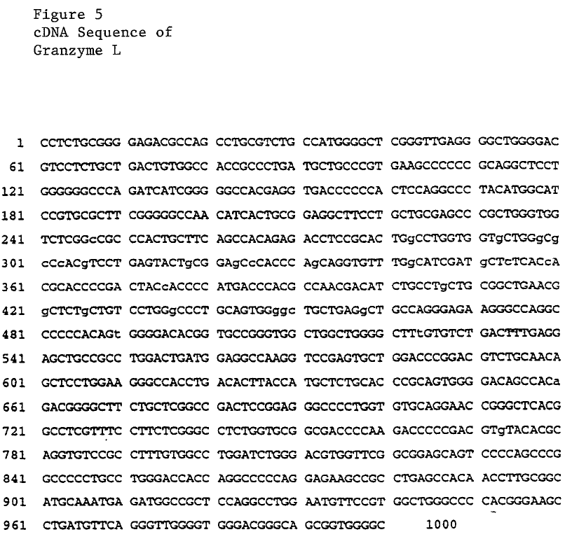

The precipitation formed was removed through centrifuging and subsequent filtration. The soluble, folded zymogen was enzymatically inactive (FIG. 4) and was purified of contaminating E. Coli proteins through cation-exchange chromatography and concentrated. A NaCl gradient of the physiological NaCl concentration was run in PBS (137 mM) up to 2 M.

### EXAMPLE 4

### The Activation (Conversion) of the Serine Protease(s) to Be Produced or the Conversion of Inactive Variants of the Serine Protease(s)

First, the activation conditions of bovine cathepsin C, of the exopeptidase to be used, were optimized.

Cathepsin C was activated in the presence of a thiol component and halidiones through reduction, e.g., with 10 mM thiolethanol amine HCl. Because the disulfide bridges of the folded granzyme may be reduced by the presence of a reduction agent in the conversion batch, however, the activation and conversion conditions were first optimized with regard to the thiol concentration, the duration of the activation and the subsequent dialysis, as well as the pH value. As an optimum parameter, 2 mM thiolethanol amine were used for the activation at a pH of 5.0 for 20 minutes, then dialyzed for 20 minutes against PBS, 75 mM Na acetate with a pH of 5.5.

The FPLC peak fraction was dialyzed against PBS pH 6.0 and concentrated to about 1 ml (˜20 mg protein/ml). Three units of cathepsin C per milliliter [stock: 5U/ml in H₂O] were first activated for 20 minutes at 37° C. in 5 mM of 2-thiolethanol amine, PBS pH 5.0, then dialyzed for 20 minutes at room temperature against PBS, 75 mM Na acetate, pH 5.5 for removing 2-thiolethanol amine. The active cathepsin C was added to the zymogen in a 1:1 ratio (Vol/Vol) and incubated for 6 hours at room temperature; formed precipitation was separated out through centrifuging, and the filtered sample was again subjected to a cation exchange chromatography procedure (see Example 3) for separating out the cathepsin C.

In activity assays, the enzymatic activity of the processed granzymes was demonstrated relative to the synthetic substrates benzyloxycarbonyl-L-lysine-thiobenzylester (Z-Lys-SBzl) for granzyme K and t-butyloxycarbonyl-Ala-Ala-Met-thiobenzylester (Boc-Ala-Ala-Met-SBzl) for granzyme M (FIG. 4). These activity tests were performed in wells of a 96-cup tray with respectively 150 μl of the sample volume.

The thiobenzylester substrates and Ellman's reagency were diluted to end concentrations of 0.3 mM in the test buffer (150 mM Tris, 50 mM NaCl, 0.01% Triton X-100, pH 7.6). The various granzyme preparations were likewise diluted to 3-15 nM in the test buffer. The color change occurring with the conversion of the substrates was measured at 405 nm and room temperature over 5 minutes in the ELISA reader. The difference in absorptions at the beginning and after 5 minutes as it relates to time was used to calculate the conversion rate.

The extent of the conversion into the desired end product was checked through amino-terminal protein sequencing (Edmann degradation), and proved to be over 90%. Similar research was conducted with fusion proteins using other helper sequences (e.g., Met-Gly-Glu-GzmK). These tests showed that different undesirable by-products (Met-Gly-Glu-GzmK, Gly-Glu-GzmK, Glu-GzmK) were present in considerable quantities in addition to the actual target substance (GzmK), and a biochemical separation of these by-products was not possible.

Yield for human granzyme K per liter E. coli culture:

| | | |
|---|---|---|
| Solubilized protein from IB: | 50-75 | mg/l |
| Folded zymogen: | 10-15 | mg/l (20%)* |
| Active granzyme: | 6-8 | mg/l (10-12%)* |

*Percentage information relates to the theoretical maximum yield of quantitatively renatured protein (= 100%)

Reference is made to Wilharm, Elke et al., “Generation of Catalytically Active Granzyme K from Escherichia coli Inclusion Bodies and Identification of Efficient Granzyme K Inhibitors in Human Plasma,” J. Biol. Chem., 274:27331-27337 (1999), the entire contents of which is incorporated herein by reference.

### Inhibition of Human GzmK by Various Inhibitors

Remaining activities of 3 nM human GzmK after incubation, with various inhibitors in percentage of the initial activity with the given concentrations. After a 60-minute preincubation at RT the residual activity was ascertained in triple batches. Initial activities were determined in buffers containing the same proportion of organic solvents. Inhibitor-enzyme quotients (I:E) were calculated on the basis of active GzmK with a titrated, active center. Molar concentrations of active aprotinine were ascertained with the use of activity-titrated bovine trypsin. CMK, chloromethylketone; TPCK, N-tosylphenylalanine chloromethylketone; TLCK, N-tosyllysine chloromethylketone; PMSF, phenylmethylsulfonyl fluoride. The results are provided in Table 1.

| | | | Residual Activity (%) | |
|---|---|---|---|---|
| Inhibitor | Conc. [mM] | I:E [× 10³] | This study | Babé et al., Bio. Appl. Bioch. 27:117-124 (1998) |
| Aprotinine | 0.015 | 5 | 8.6 | 0 |
| Benzamidine | 27.0 | 9000 | 43.1 | 50 |
| Leupeptin | 0.2 | 67 | 78.0 | 50 |
| Pepstatin A | 0.001 | 0.3 | 93.0 | 100 |
| EDTA | 10.0 | 3300 | 106.4 | 100 |
| PMSF | 2.0 | 670 | 0.0 | 0 |
| PefablocSC | 0.5 | 170 | 26.0 | |
| | 1.0 | 330 | 5.7 | |
| EGR-CMK | 0.1 | 33 | 548 | |
| FPR-CMK | 0.1 | 33 | 0.0 | |
| TLCK | 0.1 | 33 | 91.0 | 0 |
| TPCK | 0.05 | 17 | 96.0 | 95 |

We claim:

1. A method for producing one or more serine proteases and/or one or more segments of one or more serine proteases in a prokaryotic host, characterized in that
(a) the serine protease(s) and/or the segment(s) of the serine protease(s) is or are expressed as recombinant protein(s) with a helper sequence at the N-terminus, with the helper sequence including at least one dipeptide having the dipeptide sequence Met-Y, and with Y being an arbitrary amino acid, with the exception of proline;
(b) the recombinant protein(s) according to (a) is or are renatured; and
(c) the helper sequence(s) of the recombinant protein(s) obtained in accordance with (b) is or are cleaved by one or more exopeptidases.

2. A method for producing one or more serine proteases and/or one or more segments of one or more serine proteases in a prokaryotic host, characterized in that the helper sequence is at least one dipeptide having the dipeptide sequence Met-Y, with Y being an arbitrary amino acid, with the exception of proline.

3. The method according to claim 1, characterized in that one or more amino acids is or are added at the N- and/or C-terminus of the helper sequence, which includes at least one dipeptide with the dipeptide sequence Met-Y, with Y being an arbitrary amino acid, with the exception of proline.

4. The method according to claim 3, characterized in that the helper sequence having at least one dipeptide with the dipeptide sequence Met-Y, with Y being an arbitrary amino acid, with the exception of proline, has one or more methionine amino acids at the N- or C-terminus of the dipeptide(s).

5. The method according to one of the foregoing claims, characterized in that the helper sequence comprising the dipeptide Met-Y or (Met)n-Y, possibly followed by further dipeptides that can be cleaved by cathepsin C, or multiples of such dipeptides in various combinations.

6. The method according to one of the foregoing claims, characterized in that the serine protease(s) and/or the segment(s) of the serine protease(s) of the recombinant protein(s) is or are an allel, a derivative, or a functionally altered or sequentially altered mutant of a native serine protease sequence.

7. The method according to one of the foregoing claims, characterized in that one or more monopeptidyl exopeptidases and/or one or more dipeptidyl exopeptidases is or are used in method step (c).

8. The method according to the foregoing claims, wherein the diamino exopeptidase is cathepsin C.

9. The method according to the foregoing claims, wherein the exopeptidase does not irreversibly alter the serine protease to be produced and/or its fragments.

10. The method according to one of the foregoing claims, wherein the fragment to be produced is the catalytic domain of a serine protease.

11. The method according to one of the foregoing claims, wherein the biologically active serine protease comprises one or more catalytic domains.

12. The method according to one of the foregoing claims, wherein the serine protease is leukocyte elastase, proteinase 3, complement factor D, azurocidine, mastocyte chymase, mastocyte tryptase, a tissue kallikrein, cathepsin G or a granzyme.

13. The method according to the foregoing claims, wherein the granzyme is granzyme A, B, K, M or L.

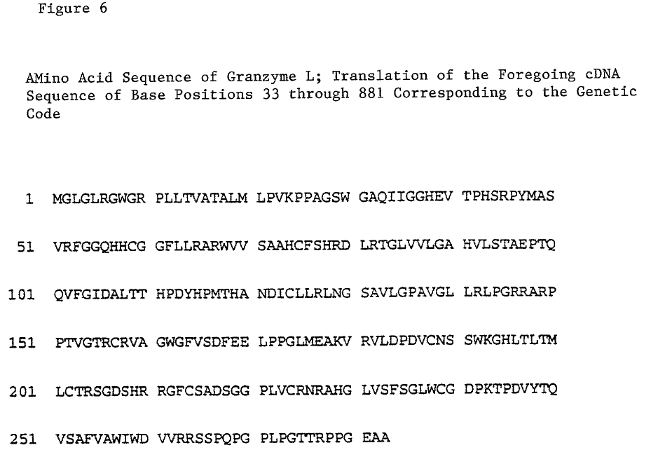

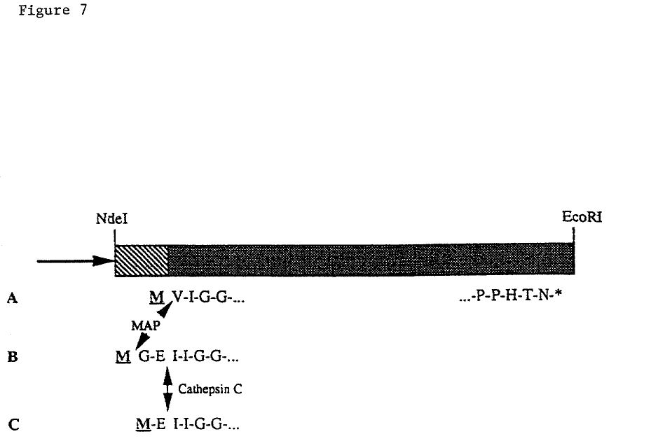

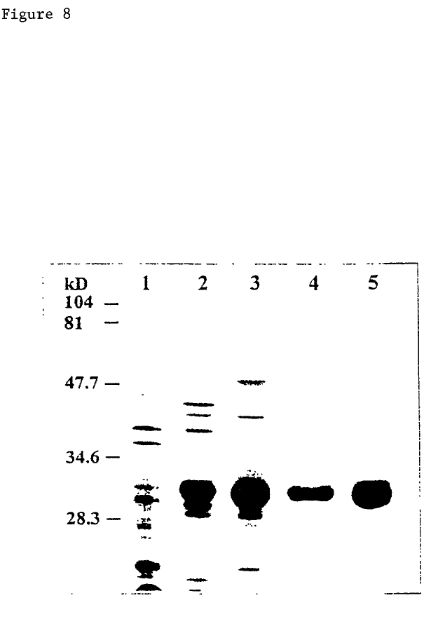

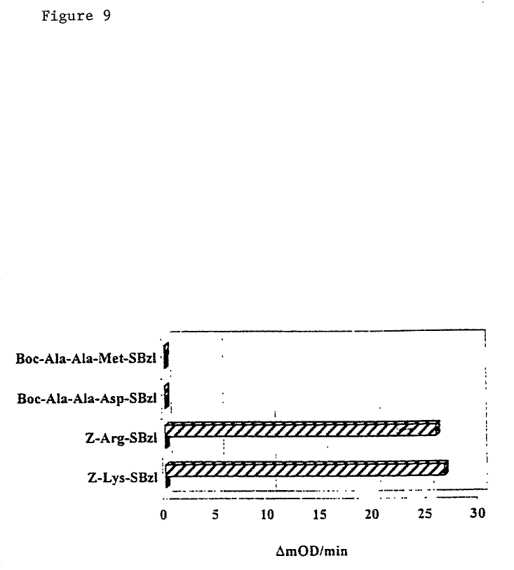

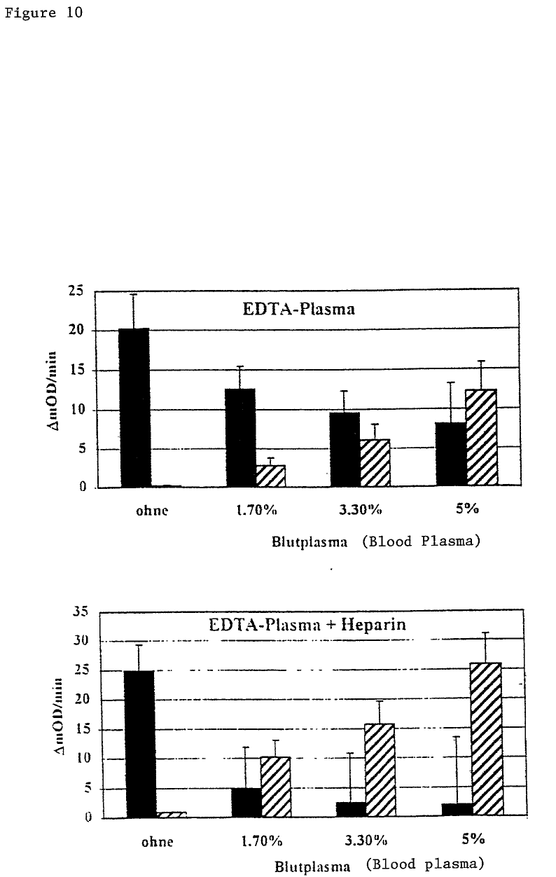

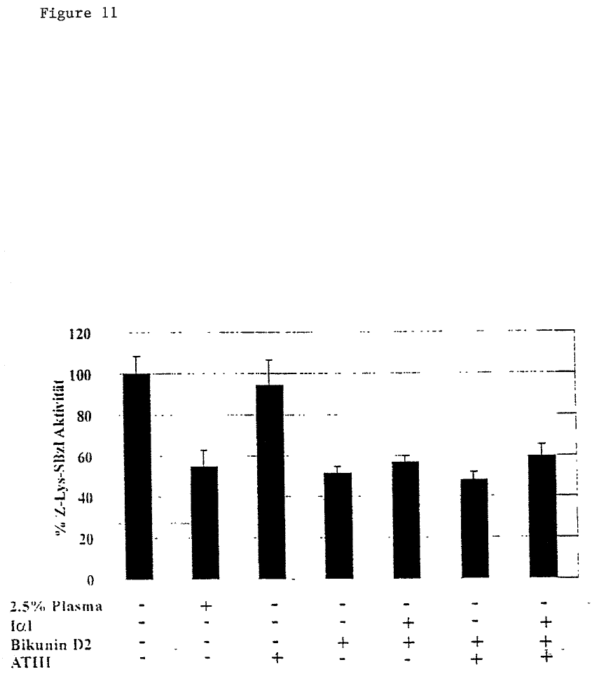

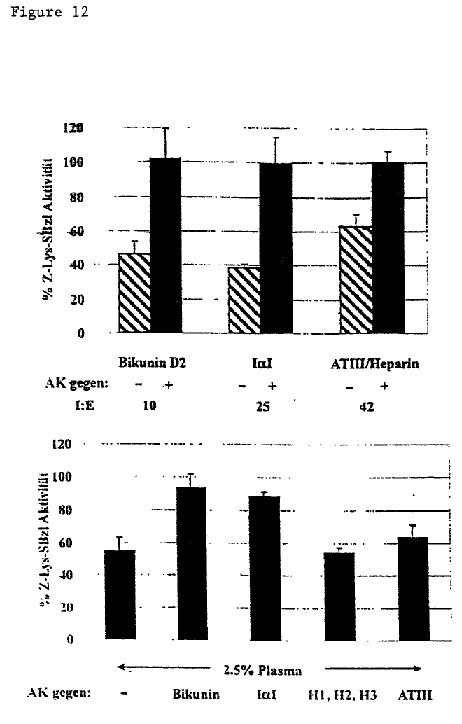

14. The method according to the foregoing claims, wherein the granzyme L includes the nucleotide sequence shown in FIG. 6, or the amino acid sequence shown in FIG. 7.
